(19) United States
(12) Patent Application Publication
(10) Pub. No.: US 2003/0096321 A1
(43) Pub. Date: May 22, 2003

(54) Method for the identification and/or the quantification of a target compound obtained from a biological sample upon chips

(76) Inventors: Jose Remacle, Malonne (BE); Joseph Demarteau, Namur (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/189,288

(22) Filed: Jul. 1, 2002

Related U.S. Application Data
(63) Continuation-in-part of application No. 09/574,626, filed on May 19, 2000.

(30) Foreign Application Priority Data
May 19, 1999 (EP) 99870106.4
Feb. 18, 2000 (EP) 00870025.4

(51) Int. Cl. G01N 33/53; G01N 33/542; C12Q 1/26; C12Q 1/68

(52) U.S. Cl. 435/7.9; 435/25; 435/6

(57) ABSTRACT

The present invention is related to a method for the identification and/or the quantification of a target compound obtained from a sample, preferably a biological sample, comprising the steps of putting into contact the target compound with a capture molecule in order in order to allow a specific binding between said target compound with a capture molecule, said capture molecule being fixed upon a surface of a solid support according to an array comprising a density of at least 20 discrete regions per cm², each of said discrete regions being fixed with one species of capture molecules, performing a reaction leading to a precipitate formed at the location of said binding, determining the possible presence of precipitate(s) in discrete region(s), and correlating the presence of the precipitate(s) at the discrete region(s) with the identification and/or a quantification of said target compound.

### CROSS-REFERENCE TO RELATED APPLICATIONS

This application is a Continuation-in-Part of U.S. patent application Ser. No. 09/574,626, filed May 19, 2000, now pending, which claims priority to European Application No. 99870106.4, filed May 19, 1999 and to European Application No. 00870025.4, filed Feb. 18, 2000.

### FIELD OF THE INVENTION

The present invention is related to a method for the identification and/or the quantification of a target compound obtained from a biological sample by binding to a capture molecule fixed upon chips.

The present invention is also related to an identification and/or quantification apparatus based upon said method, that allows the identification and/or the quantification of positive locations of bounded target compounds upon said chips.

### BACKGROUND OF THE INVENTION

Biological assays are mainly based upon interaction specificity between two biological molecules such two strands of nucleic acid molecules, an antigen and an antibody or a ligand and its receptor. The present challenge of biological assays is to perform simultaneously the multiple detection of molecules present in a sample. Miniaturization and development of arrays upon the surface of “biochips” are tools that allow multiplex reactions in a microscopic format, said detection being made with a limited volume of sample for the screening and/or the identification of multiple possible target compounds. These arrays are formed of discrete regions, containing a specific capture molecule used for the binding of the target compound. These discrete regions, as small as a few micrometers, allow the fixation of several thousands capture molecules per cm² surface (WO 95/11995).

However, the detection of bounded target compounds is difficult, since their amount is very small due to said miniaturization (few fentomoles or even few attomoles). Therefore, only extremely sensitive methods are adequate for such detection.

It has been proposed a labeling of a target compound like DNA with fluorescent molecules after their possible genetic amplification. When an RNA molecule has to be detected, it is first transformed into a cDNA, before its possible amplification. If direct labeling of the target compound is not possible, a double reaction (sandwich reaction) can be performed. However, the amount of fluorescent molecules is so low that it is necessary to develop specific array scanners for the detection and/or the quantification of the bounded compound upon the “hybridization chips”. Said expensive specific scanners comprise a laser scanner for excitation of the fluorescent molecules, a pinhole for decreasing the noise fluorescent background, and a photo-multiplier for increasing the sensitivity of the detection.

It has also been proposed methods based upon the precipitation of specific products resulting of a colorimetric labeling (U.S. Pat. No. 5,270,167) or the result of an enzymatic activity (WO 86/02733). However, said methods are either characterized by a low sensitivity or are not adequate for the detection of a target compound upon “hybridization chips”, because the precipitate will occur at a certain distance of the reaction binding and its location can not be easily correlated with a specific bounded target compound. In addition, the density of the precipitate of such enzymatic reactions is not enough opaque for allowing a detection by light absorption.

It has also been proposed to improve the detection by fixing a soluble product obtained from the enzymatic reaction with a metal before its precipitation. However, as the result of said enzymatic reaction is a soluble product, there is no correlation between the location of the precipitate and the detection of a specific bounded target compound.

The U.S. Pat. No. 6,294,327 describes an apparatus and method for detecting samples labeled with material having strong light scattering properties by using a combination of reflection mode light and diffuse scattering.

Said apparatus and method are based upon the use of two light sources for having in a time succession both reflection and scattering measurement of the same sample and then combining the two measurements for quantification.

The U.S. Pat. No. 6,171,793 also describes a method for increasing the dynamic range of a sample using a scanner and making successively two measurements with change in one parameter and then calculate the scale factor correlation of the two data converting the first data to have the same scale factor and combining the two data to obtain the larger dynamic range. The method was developed for the fluorescence detection of microarrays where by changing for example the wavelength of the laser beam of the scanner, it is possible to quantify either the high or the low fluorescent spots.

In another patent, U.S. Pat. No. 6,214,560, analytes from a sample are detected using high scattering property of particles having size between 1 and 500 nm. In this method, the analyte is being bound in the sample with a light scattering particle and use then for the detection.

Aims of the Invention

The present invention aims to provide a new identification and/or quantification method of one or more target compounds present (possibly simultaneously) in a biological sample and that will not present the drawbacks of the state of the art.

The present invention aims to provide such a method that is simple and not expensive, that allows the detection of said target compounds by using fixed bounded capture molecules upon arrays of the surface of a solid support.

A last aim of the present invention is to provide also a simple and non-expensive apparatus based upon said method, that improves the identification and/or the quantification of bounded target compounds upon “hybridization chips”.

### SUMMARY OF THE INVENTION

The present invention is related to a method for an identification and/or quantification of at least one target compound present in a biological sample by through its binding upon a capture molecule fixed (bounded) upon arrays of a solid support (hereafter called “hybridization chips”), the binding of said target compound upon its corresponding capture molecule resulting in the formation of a metallic precipitate (metal deposit) at the location of said capture molecule.

Advantageously, said method comprises the steps of:

putting into contact a target compound with a capture molecule in order to allow a specific binding between said target compound with a (corresponding) capture molecule, said capture molecule being fixed (bounded) upon a surface of a solid support according to an array comprising at least a density of 20 discrete regions per cm², each of said discrete regions being fixed (bounded) with one species of capture molecules,

performing a reaction, preferably a (chemical or biochemical) catalytic reaction, leading to a formation of a metallic precipitate (metal deposit) at the location of said binding,

determining the possible presence of a metallic precipitate (metal deposit) in a discrete region preferably by the detection and possibly recording means such as a scanner, and

correlating the presence and/or the formation of the metallic precipitate(s) at the discrete region(s) (precipitate pattern) with the identification and/or a quantification of said target compound in the biological sample.

### BRIEF DESCRIPTION OF THE DRAWINGS

FIG. 1 compares the detection of target molecules obtained on arrays composed of DNA capture nucleotide sequences covalently fixed on glass and used to detect 3 concentrations of biotinylated target DNA either in fluorescence or after silver concentration.

FIG. 2 represents the disposal of elements in the detection device according to the invention for making both Retro-diffusion (FIG. 2a) and Transmission (FIG. 2b) measurements.

FIG. 3 shows results of a measurement obtained by combination of the retro-diffusion (triangles) and the two transmissions (X).

FIG. 4 presents digitalized images from the same array of spotted DNA probes obtained with the retrodiffusion (left) or transmission (right) methods.

FIG. 5 is a schematic representation of the transmission method (light blocked by the silver spots).

FIG. 6 gives a molecular representation of the light beams into the metallic particles in the transmission mode

FIG. 7 is a schematic representation of the Retro diffusion method ( light waves are diffused by metal particles).

FIG. 8 gives a molecular representation of the light into the metallic particles in the Retro-diffusion mode.

FIG. 9 shows an example of the detection of autoimmune antibodies in serum of patients using the colorimetry detection according to the invention on protein microarrays.

FIG. 10 shows digitalized pictures of rat liver gene expression microarrays of a control rat and a phenobarbital treated rat detected in colorimetry method according to the invention.

FIG. 11 presents an automate (robot) for handling liquid for simultaneously processing several microarrays present on a surface.

FIG. 12 presents the location of a pipette controlled by the automate on one among the 24 array present on the same surface.

FIG. 13 is a presentation of a pipette containing a chamber on which are located the capture molecules and processes for liquid handling controlled by an automate using solutions present in a 96 well plate.

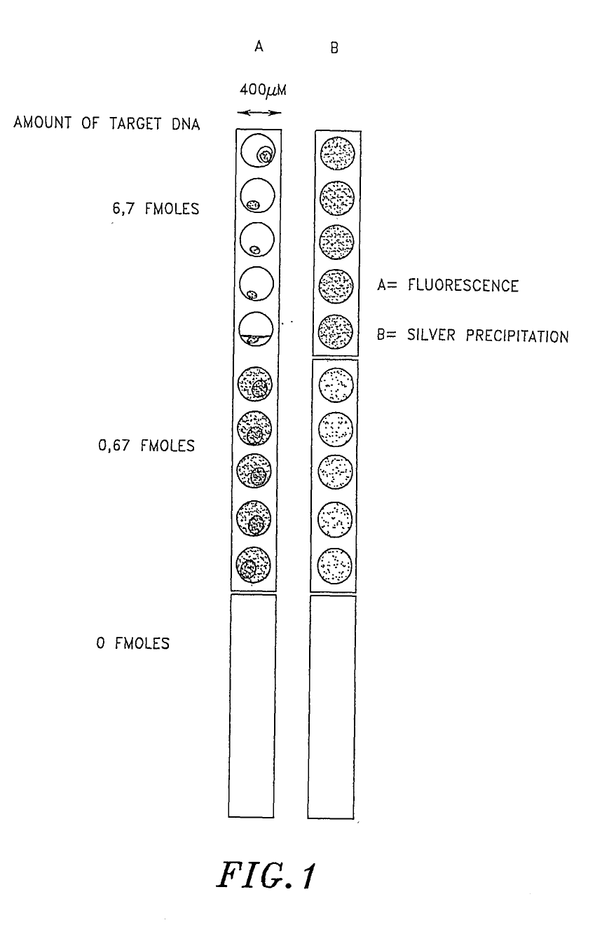

### DETAILED DESCRIPTION OF THE PREFERRED EMBODIMENT

As stated above, the present invention is related to a method for an identification and/or quantification of at least one target compound present in a biological sample by through its binding upon a capture molecule fixed (bounded) upon arrays of a solid support (hereafter called “hybridization chips”), the binding of said target compound upon its corresponding capture molecule resulting in the formation of a metallic precipitate (metal deposit) at the location of said capture molecule.

The “hybridization chips” according to the invention are any kind of solid support that allow the formation of arrays of capture molecules (specific pattern) upon one or more of its surfaces. Said solid support can be made of glasses, filters, electronic device, polymeric or metallic materials, etc., including materials such as plastic supports which present an intrinsic fluorescence. Preferably, said arrays contain specific locations (advantageously presented according to a specific pattern), each of them containing normally only one species of capture molecule.

The fixation (binding) of DNA strands on proteins thereafter specifically attached to sites specific locations on a substrate, is described in the document U.S. Pat. No. 5,561,071. It is also known that capture chemicals can be linked to microtubes that are then spatially arranged in order to produce an array, as described in the document GB-3 319 838, or to obtain the direct synthesis of oligonucleotides on specific surfaces by using photolithographic techniques as described in the documents WO 97/29212 and U.S. Pat. No. 5,632,957.

All these methods for the fixation (binding) of capture molecules on the surface of a solid support in order to obtain the above-described arrays are compatible with the present invention.

The biological target compounds according to the invention may be present in a biological (or possibly a non-biological) sample such as possibly purified clinical samples extracted from blood, urine, feces, saliva, pus, serum, tissues, fermentation solutions or culture media. Said target compounds are preferably isolated, purified, cleaved, copied and/or genetically amplified, if necessary, by known methods by the person skilled in the art, before their detection and/or quantification upon the “hybridization chips”.

Preferably, the formation of a metallic precipitate at the location of the binding is obtained with the fixation of a metallic compound upon the (bounded) target compound or by the result of a metal precipitation in the presence of an enzyme. Advantageously, a reduction of silver in the presence of colloidal gold allows the formation of a precipitate (metallic deposit) at a distance not exceeding few micrometers from the bounded target compound to its corresponding capture molecule.

According to the invention, the specific locations on the array are smaller than 1000 μm in length. These locations or spots have preferably a diameter comprised between about 10 and about 500 μm and are separated by distance of similar order of magnitude, so that the array of the solid support comprises between about 100 and about 250,000 spots upon the surface of 1 cm². However, it is also possible to prepare spots smaller as 1 μm or less upon which the capture molecules are fixed. The formation of said spots or locations would be obtained by known microelectronic or photolithographic processes and devices that allow the fixation (binding) of said capture molecules on the surface of the solid support either by a covalent linkage or a non-covalent adsorption. The covalent linkage technique is preferred in order to control specifically the sites of capture molecules fixation and avoid possible drawbacks that may result with several capture molecules (like nucleic acids or antibodies) that can be desorbed during incubation or washing step.

One of the preferred embodiment is the fixation (binding) of biological molecules like proteins, peptides, sugars or nucleic acid sequences by linkage of amino groups on activated glass (solid support) bearing aldehyde moiety. The incorporation of an amine group in the nucleic acid chain is easily obtained using aminated nucleotide during their synthesis. Aminated amino acids can be fixed upon the surface of a solid support like glass bearing aldehyde groups as described by Schena et al. (Proc. Natl. Acad. Sci. USA, 93, pp. 10614-10619 (1996)) or as described in the document U.S. Pat. No. 5,605,662 and the publication of Krensky et al. (Nucleic Acids Research, 15, pp. 2891-2909 (1987)). The linkage between an amino and a carboxyl group is obtained by the presence of a coupling agent like carbodiimide compounds as described by Joos et al. (Anal. Biochem., 247, pp. 96-101 (1997)). Amino groups also form covalent links with other chemical reactive groups such as epoxide, acrylate, alkyl halide, acylhalide, isocyanate or thiocyanate. Thiol modified oligonucleotides can be used also to obtain a reaction with amino groups upon the surface of a solid support in the presence of cross-linking molecules (Thrisey et al., Nucleic Acids Research, 24, pp. 3031-3039 (1996)). Similarly, oligonucleotides can be fixed to a gel like polyacrylamide bearing hydroxyl and aldehyde groups as described in the document U.S. Pat. No. 5,552,270 and WO 98/28444. Sugars such as polysaccharides or sugar bearing proteins are best fixed after periodate oxidation into dialdehyde and then fixation on aminated surface.

Polyvinyl or polyacrylic polymers bearing or containing in the resin chemical reactive groups such as aldehyde, epoxide, acrylate, hydrazine, thiocyanate can be used according to the invention. One particular useful method is the grafting or coating of a polyacrylate polymer containing aldehyde groups by incorporation of glycidyl methacrylate such as described by Eckert et al. (Biomaterials, 2000, 21, p.441). Polymers bearing reactive groups are possibly coated on any surfaces such as glass, metal or plastic making then available as microarray supports.

Polymers such as polyolefine, polyvinyl, polyacrylique, polymethylmethacrylate bearing or containing in the resin chemical reactive groups such as aldehyde, epoxide, acrylate, hydrazine, thiocyanate are also an embodiment of this invention. Polymers bearing reactive groups are possibly coated on any surfaces such as glass, metal or plastic making then available for microarray supports. Of particular interest is the use of spin coating and radcure radiation for the formation of a polymer onto the surface of the support while incorporating chemicals with reactive groups for capture probe fixation. One of such chemicals is epoximethacrylate which incorporates into the polymer chain through its vinyl group but keep its epoxide group reactive for the further fixation of capture nucleotide sequences.

According to a preferred embodiment of the present invention, the binding of the capture molecules upon the surface of the solid support is obtained according to the method described in the document W002/18288 incorporated herein by reference.

The binding (or recognition) of the target compound upon corresponding specific capture molecules may be a spontaneous non-covalent reaction when performed in optimal conditions. It involves non-covalent chemical bindings. The medium composition and other physical and chemical factors affect the rate and the strength of the binding. For example for nucleotide strand recognition, low stringency and high temperature lower the rate and the strength of the binding between the two complementary strands. However, they also very much lower the non-specific binding between two strands (which are not perfectly complementary). When several sequences are similar, the specificity of the binding can be enhanced by addition of a small amount of non-labeled molecules, which will compete with their complementary sequence, but much more with the other ones, thus lowering the level of cross-reactions.

The optimization of the binding conditions is also necessary for antigen/antibody or ligand/receptors, chemical-enzymes recognition, but they are usually rather specific.

In a particular embodiment the target compound is identified and/or quantified according to a signal characteristic of cell activation. Cell activation include a large range of processes (among which phosphorylation, acetylation or methylation) leading to the presence of new phosphate, acetyl or methyl groups on proteins, DNA or sugars. The presence of these groups is best obtained by the use of antibodies specific of the presence of such groups in particular locations of the proteins, DNA or sugars.

In another embodiment the detected target protein is detected after interaction with another molecules bound to the support either directly or through another molecule. Of particular interest is the use of antibodies to immobilize one particular protein and to screen for the presence in a sample for other proteins which interact with the immobilized first protein.

A preferred embodiment of this invention is to take party of the amplification given by the catalytic reduction of Ag⁺ in the contact of other metals like gold. Gold nanoparticules are currently available and they can be easily fixed (bounded) to molecules like protein. For example, streptavidin and antibodies coated gold particles are available on the market (BBI International, Cardiff, England).

According to a preferred embodiment of this invention, one uses a labeled target molecule, which is then recognized by a conjugate. This labeled molecule (e.g., biotin, haptens, etc.) can be considered as a first member of the binding pair. For DNA, the labeling is easily done by incorporation of biotinylated nucleotides during their amplification. For the RNA, biotinylated nucleotides are used for their copy in cDNA or thereafter in the amplification step. Amplification of the nucleotide sequences is a common practice since the target molecules are often present in very low concentrations. Proteins are easily labeled using NHS-biotin or other reactions. Once the biotinylated molecules are captured, a streptavidin-gold complex, which is the second member of the binding pair, is added and the streptavidin specifically recognizes biotin, so that the complex is fixed at the location where the target is fixed. If haptens are used as label, an antibody-gold complex will be used.

One may use also biotinylated molecules target or reagents recognized thereafter by specific antibodies-gold complex. Then a reactive mixture containing Ag⁺ and a reducing agent is added on the surface and Ag layers will precipitate on the gold particles leading to the formation of crystal particles. Hydroquinone is the preferred reducing agent for metal precipitation but other reducing agents used in the photographic process are other choices to form silver crystals.

Direct labeling of the target molecules with gold is possible by using gold-labeled antigens, antibodies or nucleotides.

An alternative is to avoid any labeling of the target molecule, and then a second nucleotide sequence is used which is labeled. They then formed a sandwich hybridization or a sandwich reaction with the capture molecule fixing the target and the labeled nucleotide sequence, which allows the detection to go on. Like above, the labeled nucleotide sequence is able to catalyze itself the precipitation of the metal or it does it through a second complex.

The Ag precipitation corresponds to the location of the binding of biotinylated nucleotide sequence. As said location is well defined, it is possible to identify the presence of said precipitate (specific spot of the array).

The precipitate has the form of small crystals that reach with time a diameter of about 1 μm. The formation of these small crystals represents a real amplification of the signal since they originated from the presence of gold particles a few nm in diameter.

Unexpectedly, within a given range of labeled nucleotide sequences present on the surface, a concentration curve could be obtained between the gold-labeled nucleotide sequence concentration and the amount of precipitate on the surface. One constraint of the array is that the detection signal has to be correlated with the location where it originates.

Because of its granular form, the precipitate advantageously modifies the reflection, transmission, (diffusion) diffraction (scattering), or absorption of the light which is recordable by known detection means. Such transmission (diffusion) assays are typically detected and recorded from the reflection of a light beam with photodiodes. One unexpected observation is that the assay for the presence of silver crystals was found unexpectedly very sensitive. Table 1 presents data on the detection of spotted solution of 5 pmoles. Since 0.5 nl were delivered per spot, this represent 2.5×10⁻² mole of nucleotide sequences present on the spot and still detectable by the present invention. Such a detection of so low concentration of DNA sequence could not be obtained by non metallic precipitate which was found around 1000 times less sensitive.

As a metal, silver is able to reflect light by itself. Because if its metal nature, other methods like variations of an electromagnetic field electric conductance or heat detection (WO 01/85978) are also possible.

According to a preferred embodiment of the present invention, the presence of deposits, specifically metal deposits, is evidence by measuring (with suitable means) its conduction of currents based on electric measurement of conductivity or resistance or impedance or any similar modification of heat or current properties obtained by the deposit of metal. Formation of the metallic precipitate is one of the application of the electric based detection since with increasing size of the precipitate the electric properties of the surface change drastically.

Preferably, metal particles are compared with the target molecules and their accessibility. The preferred particle size of metal deposits are from 1 nm to 20 nm diameter that could be as large as 100 or even 200 or more than 1000 nm in diameters or may comprise an equivalent diameter and an important volume.

According to another preferred embodiment of the present invention, the precipitate forms particles which are used for catalyzing a reaction of which the formation rate can be followed by recording means. The metal catalytic properties which are preferred are the reduction of other metals and/or the formation of a crystal deposit. Preferably, the reduction rate can be detected and recorded by measurement of electrons used in said reduction and said measurement is advantageously performed by similar amperometric measurement.

According to a preferred embodiment of the present invention, the precipitate deposit is preferably a metal deposit located between two electrodes present in the solid support or at the surface of the solid support which creates a bridge which will modify the electric properties of one or more of the electrodes, preferably a modification in the resistance or the independence which can be measured between the two electrodes.

Preferably, the metal deposit is selected in order to obtain a higher conductivity which can be easily measured, preferably, between inter-digitalized electrodes.

The preferred distance between the electrodes is between about 0.1 μm and about 1 μm but smaller distances, for instance, between about 1 and 100 nanometer can be also adapted by the person skilled in the art for specific nanomeasures and can be placed also between larger distances (from about 1 to about 10 μm). Each discrete region of the array comprising capture molecules can be of any geometrical form. Preferably, said discrete region of the micro-array comprising capture molecules lie between about 1×10⁻³ mm wide and spaces between about 1×10⁻³ and about 20 mm. Each line array being selected for comprising capture molecules specific of the target molecule and allow the specific identification of biomolecules, specific for a species, an organism, a genus family, a pathology or a group of genes. Preferably, the detection is obtained also by apparatus of a specific line by using a lecture of bar code systems.

In a preferred embodiment the present invention is related to the use of detector for imaging the sample comprising metallic precipitate by measurement of the absorption of the transmitted light through the surface of the solid support bearing the said metallic precipitate and correlating the said absorbed light with the presence of target molecules fixed on the capture molecules present on the surface. The detector preferentially detects in a statistically significant way concentrations of 3 logs or more.

A further aspect of the present invention is related to a method for imaging a sample, (preferably said solid support surface comprising said metallic precipitate) comprising projecting a transmission mode light from a (first) light source onto said sample during a transmission mode time period, detecting light on detector from said (first) light source which has been transmitted through said sample, and projecting diffuse scattering light from the same (or a second) light source onto said sample during similar or other than said reflection mode time period and detecting reemitted light on said detector from said sample. The method for imaging a sample according to the invention combines transmission and diffraction (scattering) which the unexpected property that the person skilled in the art is able to obtain by transmission a measure (detection and possibly quantification) upon the sample (spotting upon a micro-array) at high concentrations while the diffraction (scattering) allows such measure at low concentrations.

It is important to note that the present invention is based upon the combination of these two measures upon the same sample.

Preferably the method of imaging is combined with the identification and quantification method according to the invention and is used for the characterization of possible precipitate, preferably metallic precipitate in discrete regions of the solid support surface. Also the presence of the precipitate is correlated with the presence and the quantification of the target molecule in the sample through corrections and standardization using appropriated softwares.

Another aspect of the present invention concerns a diagnostic (detection) and/or quantification apparatus of one or more identical or different target compounds obtained from a sample, said apparatus comprising:

a solid support with an array surface having at least 4, preferably at least 10, more preferably at least 20 discrete regions per cm² surface, each of said region being fixed (bounded) to one species of capture molecules corresponding to (which recognizes) a target compound,

a detection and/or quantification device of metallic precipitate(s) (spots) upon the surface of said solid support resulting from a binding of said target compound upon a corresponding capture molecule,

possibly a reading device of information(s) recorded upon said solid support (such as barcodes) and

a computer programmed (configured to interact with reading device(s) to:

possibly recognize the discrete regions bearing capture molecules,

collect the results obtained from said detection and/or quantification device, possibly correlated with the information(s) obtained from said reading device, and

carry out a diagnostic and/or quantification of said target compound(s).

The present invention is also related to a device for imaging a sample preferably integrated in the apparatus according to the invention as a detection and quantification device of precipitate above-mentioned.

Preferably, said device comprises a (first) light source providing a transmission mode light to the sample, and a second or same light source providing diffuse scattering (diffraction) light to said sample, a detector and a computer programmed (configured) to interact with said detector, such that said detector detects light transmitted from said sample in response to application of light from said (first) light source and said detector detects reemitted light in response to application of light from said (second) light source wherein said device is configured to cause the (first) light source to provide a transmission mode light to the sample, preferably during a (first) time period, and to cause a (second) light source to provide diffuse scattering light to said sample (preferably during a time period other than said first time period). The emitting light at the opposite side of the camera causing the diffracted light is considered as “retro-diffusion” light.

When the background in front of the camera is white and the sample is lit by an uniform peripheral light source, then the scanning is in transmission.

When the background in front of the camera is black and the sample is lit by an uniform peripheral light source coming from behind the camera, then the scanning is in normal diffusion.

In a preferred embodiment the apparatus for detection comprises a light source obtained from a circular neon tube 3, a black background 4 and possibly a white moveable translucent surface 5 disposed between the solid support (slide sample 2) and the source light 3 or wherein the source light 3 is disposed between the solid support 2 and said white surface 5. In a more simple and preferred device, transmission of the light through the surface of bearing the capture and target molecules is measured and the transmitted light absorbed in the locations of the presence of the capture nucleotide sequences (spot) is a measure of the presence and a quantification of the bound target. The absorbed light in the locations of the capture nucleotide sequences (spots) is preferentially corrected for the background by subtracting the absorbed light in the surface locations not having capture nucleotide sequences preferentially the quantification of each spot is corrected by absorbance of the surface surrounding each spot.

In the device according to the invention, any suitable detector 1 such a diodes elements, a fiber optic bundle, a CCD camera or a CMOS camera, alone or arranged in row, of said transmitted or diffracted light can be used. Detectors such as CCD sensors are either matricial or linear.

The person skilled in the art is also able to provide means for performing the various steps of the present invention, especially the transformation and the conversion of the measure into a digital form or a set of digital forms by using known means or methods such as the ones existing in software and computer technologies.

The device for imaging a sample according to the invention comprises also a carrier element for supporting a sample. Said sample is preferably a transparent polymeric or a glass slide and said support is configured for allowing the introduction of the sample into the opening (bay) of the device (scanner or detector apparatus, possibly integrated in the case of a personal computer according to the invention). Said carrier having a size suitable for carrying one slide, comprises attaching means and a (preferably central) transparent or open window allowing the transmission of the mode light from the first and/or second light source upon said sample.

In a particular application, the formation of the precipitate is follow by the detection device and the kinetic of the formation of the precipitate transformed into a quantification of the present target on the support.

The method and apparatus according to the invention are suitable for the high-throughput screening of target compounds, possibly present in multiple samples.

Therefore, in the high-throughput screening method and apparatus according to the invention, the solid support may comprises between 4 and 1536 arrays disposed according to a pattern of a multiple well microtitre plate 10. The arrays are disposed in a rectangular pattern according to the disposition of the wells of a 24, 96, 384 or 1536 microtitre plate format, preferably of the 96 well plate format having 8 rows large and 12 rows long or multiple wells titer plate having a similar configuration. The microarrays are disposed in a pattern that can be superposed to the locations of the wells of these plates with possibly some locations being empty or possibly arrays recovering two or more locations.

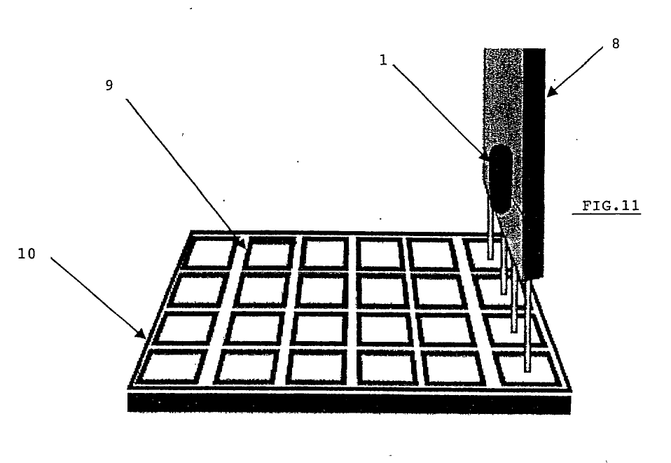

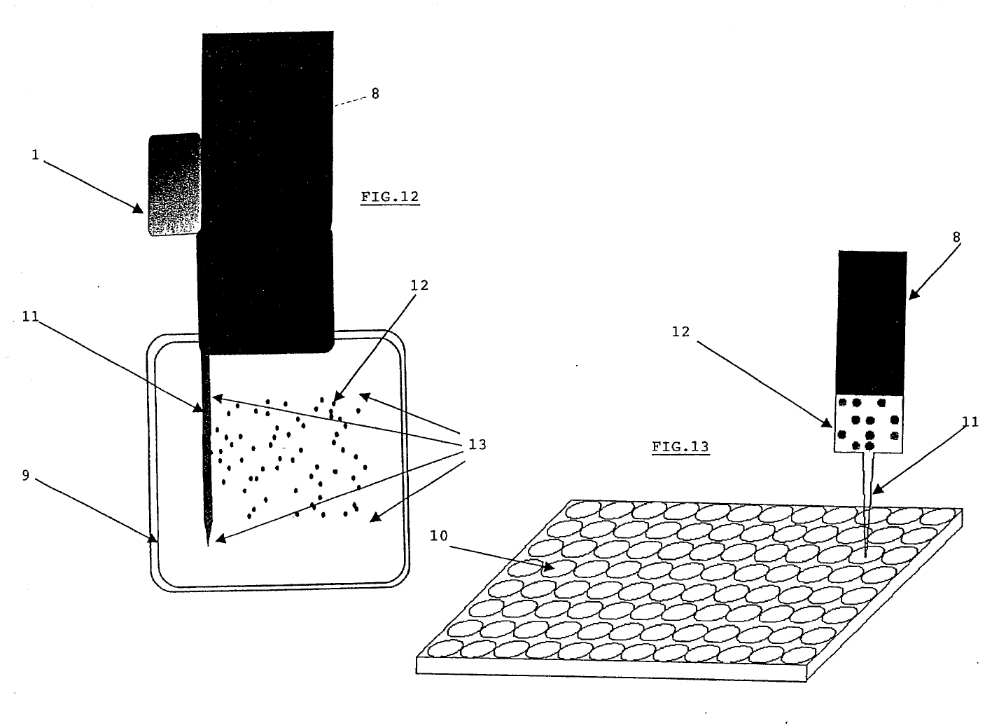

In the method according to the invention, the sample comprising the target compound(s) to be detected and/or quantified are handled by automatic injection and aspiration means (micropipettes 11). Also, the solutions for washing or labeling the target present on the arrays are handled by automatic injection and aspiration means (FIGS. 11 and 12).

In the preferred embodiment, said injection and aspiration means (pipettes) 11 and detectors 1 are disposed in lines of 8 or 12 in order to handle consecutively and automatically the injection and aspiration of the sample and various media and allow a detection and/or quantification according to the invention. The aspiration and injection device are preferably present on a moving arm 8 (of an automate) which cover the overall plate 10 and moves at least according to X/Y axes of said solid support surface for delivering the solutions at the appropriated locations 13.

According to an alternative embodiment of the present invention, the injection and aspiration means are static and it is the solid support 9,10 of said microarray 12 which moves according to each processing step of the method according to the invention.

In the high-throughput screening method and apparatus according to the invention, the various micro-arrays are disposed upon a planar element having a rectangular surface with 8 rows large and 12 rows long, each row comprising one or more different or similar microarrays. The overall distance between the center of 2 microarrays is usually comprised between about 5 mm and about 5 cm.

The distance between adjacent wells is usually 9 mm. For formats derived from for this reference, the inter-well distance of 9 mm is divided by the miniaturization factor, which is defined as:

$$m = \sqrt{\frac{n\_wells}{96}}$$

with n-wells being the number of wells.

The format of the obtained microarrays wells could be made in any type of material such as but not limited to metal, steel, silicon, silicon oxide, silicon nitride, silicon oxynitride, polysilicon, porous silicon, plastic, polymer (including rubber, PVC, etc) biodegradable polymer, glass, quartz, ceramics, aluminum oxide, nitrocellulose, nylon or some specific biological material.

In a preferred embodiment, said microarrays are recovered by a (possibly closed) incubation chamber 9 which is possibly removed during one or more processing step(s). Automatic pipeting is then performed within a location 13 inside the chambers 9.

The format of standard microtitre plate 10 are but not limited to 24-wells, 96-wells, 384-wells, or 1536-well microtitre plates, customized for integration in any suitable high-throughput screening systems. A robotic comprising suitable dispensing and titer plate handling.

In a preferred embodiment the apparatus comprises an automatic liquid handling device 8 for pipeting in the array(s) and a detection and/or quantification 1 device of the precipitate.

Preferentially the automate delivers solution through 1 to 96 or even 384 pipettes present on a moving arm and dispensing liquid volumes from 1 μl to 1 ml delivered in the microarray chambers. The automate dispenses solution in positions compatible with either 96 and 384 well plates. The robot is well adapted to high-throughput operations: dispensing or pumping liquid by pipette of an arm in 96 microarrays is done in less than 10 seconds. Ten plates can be processed during the same run. Stacker allows to place more plates for multi-runs.

In one particular embodiment the detector 1 and the surface of the array(s) move comparative to each other in a perpendicular X and/or Y axes (of the solid support surface) relative to each other. Still the automatic pipeting and/or detector support comprises an automatic arm 8 having said X and/or Y movement pattern according to steps of 9 mm or a multiple of it. In a preferred embodiment one or more CCD camera 1 are present on the arm 8 of the automate for performing successive detection of each of the array 12 present on the support 9,10.

Detection of the microarrays is performed simultaneously or consecutively by a computer controlled moving device which allows an analysis of each array present on the surface and attribute the data of the arrays to the samples initially introduced in such array.

In a particular embodiment of the invention well adapted for high throughput analysis, the support 12 bearing the capture molecules is inserted or is part of the pipette 11 (see FIG. 13). Being detected by colorimetric method, pipette 11 or part of the pipette bearing the capture molecules 12 is made of material transparent to light preferentially polymer material such as polypropylene coated or modified as explained here above for the fixation of capture molecules. Preferentially the tip of the pipette is round and follow by a square or round part on which is fixed the capture molecules. The support bearing the capture molecule can also be inserted as a separated material inside the pipette. The pipette incorporated capture molecules (preferentially under the form of (micro)array) is then adapted to a pipeting machine or automate in order to perform the various steps according to the invention: pipeting of the sample, washing by solutions and buffer adding calorimetric reagents. The method is particularly well adapted for high throughput screening on microarrays using 96, 384 or even 1536 multiwell plates 10 containing the solutions for performing the various steps of the process. The microarray is then detected according to one of the detection process explained here above or any other ones and data analyzed for the presence and/or quantification of the target(s) molecules. Preferentially the (micro)array-pipette is manufactured by application of a polymer surface bearing the capture molecules on a frame present on the micropipette and sealing the two to make them impermeable to water while creating a chamber 9 between the two surfaces.

The present invention is also related to a computer program product (software) comprising program code means configured for performing or controlling all or part of the step of the method according to the invention, when said program is run on a computer and interact with the detector and/or reading device.

The present invention is related to a computer program product comprising program code means stored on a computer readable medium and configured for performing or controlling the method according to the invention, when said program product is run on a computer and interact with the detector and/or reading device.

Said means are able to collect the results obtained from said detection and/or quantification device and possibly the information(s) obtained by said reading device, and said means are able to carry out a diagnostic and/or quantification of a specific target compound resulting from the analysis of said results, possibly correlated to the read information(s) and attribute said results to a specific sample tested according to the method of the invention.

Said means of this computer program product are able to obtain a discrimination between the spots and a possible detected background noise, for instance by the identification of homogeneous parts of an image after having been merged into two classes used as training sets. This discrimination can be enhanced by post-classification contextual filters techniques.

Said means are also able to identify the contour of the spot itself, which will be superposed to the original image and will allow the measure of intensity level of the counted pixels identified in the spot.

The quantification means allow an integration of all pixels intensity present in the spot or a recording the overall level of intensity of the homogeneous parts of the spot.

Furthermore, these means allow a statistical comparative analysis between the spots of each sample and a control or reference standard (standard target compound) or between two or more spots (preferably with a correlation with the recorded information of the solid support). Image correlation could be obtained between the spot image and said standard target compound spot image in order to discriminate spots that are statistically different in one test compared to another. The different targets of a sample which amounts are statistically different from a reference sample represents a pattern of targets typical of the said sample. A modified pattern in gene expression or protein content determined according to the method of the invention is one particular useful embodiment of the invention

The recorded signal(s) by the detection device and the reading device can be read, processed as electronically computerized data, analyzed by said appropriate computer program product (software).

According to a specific embodiment of the present invention, the array bears fixed (bound) oligonucleotide capture nucleotide sequences so as to allow a detection, amplification and possibility quantification of nucleic acid sequences upon a same solid support. In an alternative form of execution, the array comprises fixed PCR primers in order to obtain the production of amplicons and fixation of amplicons upon the surface according to the method described by Rasmussen et al. (Anal. Biochem., 198, pp. 138-205 (1991)), which allows thereafter their detection.

The array according to this invention is used in a diagnostic kit, in a diagnostic and/or quantification apparatus which allows automatic lecture, possibly after a previous treatment, such as purification, cleaving, copying and/or genetic amplification.

Preferably, the detection and/or quantification apparatus according to the invention is a system that combines multiple steps or substeps within an integrated system as an automatic nucleic acid diagnostic system (the steps of purification of the nucleic acid sequences in a sample, of amplification (through known genetic amplification methods), the diagnostic and possibly the quantification).

Preferred embodiments of the present invention will be described in the following non-limiting examples in reference to the figures.

### EXAMPLE 1

### Detection of DNA on Biochips

In this experiment, target DNA labeled is detected by direct hybridization on capture nucleotide sequences bound to the array. Capture nucleotide sequences were covalently bound on glass and direct hybridization performed with complementary biotinylated DNA. The positive hybridization was detected with silver precipitate catalyzed by the nanogold particles linked to streptavidin.

Binding of Capture Nucleotide Sequences on Glass

Activated glass bearing aldehyde groups were purchased from CEL Associates (USA). Aminated capture nucleotide sequences for CMV DNA were constructed by PCR amplification of the DNA using aminated primer as described by Zammatteo et al. (Anal. Biochem., 253, pp. 180-189 (1997)). The primers were purchased from Eurogentec (Liege, Belgium). Quantification of the amplicons was done by their absorption at 260 nm.

For the grafting on glass, a solution of aminated amplicons at 0.2 μm in MES 0.1 M pH 6.5 was first heated at 100° C. for 5 min and then spotted by a robot using 250 μm diameter pins (Genetix, UK). After incubation of 1 h at 20° C., they were washed with SDS solution at 0.1% and then two times with water. They were then incubated with NaBH₄ at 2.5 mg/ml solution for 5 min then washed in water and heated at 95° C. for 3 min before being dried.

Hybridization of the Target Molecule

The target molecule was obtained by amplification by PCR in the presence of biotinylated dUTP at 1 mM (Alexandre et al., Biotechniques, 25, pp. 676-683 (1998)). Plasmids containing the sequence of CMV virus were used for the PCR. After amplification, the PCR products were purified using a kit of high pure PCR product purification (Boehringer, Mannheim, Germany) and quantified by ethidium bromide staining after separation on a 2% agarose gel.

For the hybridization, various concentrations 0.67, 6.7 and 67 fin in 5 μl of biotinylated target DNA were added in a SSC 2× Denhard solution containing 20 μg of Salmon DNA. A drop of this solution (5 μl) was added on the array and incubated for 2 h at 65° C. in a wet atmosphere. The array was then washed 4 times with a maleic acid buffer 10 mM pH 7.5, containing NaCl 15 mM and Tween 0.1%.

Silver Precipitation on the Array After Silver Precipitation

The array was first incubated for 45 min with 0.8 ml of a streptavidin-colloidal gold (Sigma) diluted 1,000 times in a maleic buffer 150 mM pH 7.4 containing NaCl 100 mM and 0.1% dry milk ponder. The arrays were then washed 5 times 2 min in the maleic acid buffer 10 mM pH 7.4 containing 15 mM NaCl and Tween 0.1%. A “silver enhancement reagent” (40 ill) from Sigma was added onto the array and changed after 10 and then 5 min. After washing in the maleic buffer, the array was dried.

Detection and Analysis of the Array

The array was scanned and the digitalized image was treated with form recognition software in order to delimitate and identify the spots. The level of the pixels of each spot was integrated and a value given to each spot. The values were corrected for the background obtained in three places where no capture nucleotide sequences have been fixed.

### EXAMPLE 2

### Detection of Rat Liver Gene Expression on Microarrays in Colorimetry Animal Treatment

Female Sprague-Dawley CD rats (aged 10-12 weeks) were dosed orally with 100 mg/kg per day of either Sodium Phenobarbitone (PB) or pregnenalone 16-carbonitrile (PCN) (Sigma-Aldrich Co. Poole, Dorset, UK) for 4 days. Control animals received corresponding quantities (5 ml/kg body weight) of the 0.56% (w/v) gum tragacanth vehicle. Animals were killed by decapitation and the livers immediately removed for further mRNA extraction.

Rat HepatoChips Design

Fifty-nine genes microarray Genes on the Rat HepatoChips are presented in the Table 1. The selected genes are either involved in drug metabolism or may have a potential to act as markers of toxicity. The arrays also include positive and negative controls for the hybridization process, an internal standard control and 8 housekeeping genes.

TABLE 1
Data of analysis of genes expression of liver on microarrays from
a control rat and a rat treated with phenobarbital

| Meta | | | | Control | Control | Test | Test |
|---|---|---|---|---|---|---|---|
| Column | Row | Col | Gene ID | Signal | Background | Signal | Background |
| 1 | 1 | 1 | Detection control | 63661 | 21724 | 63942 | 20549 |
| 1 | 1 | 2 | Detection control | 63569 | 24079 | 63958 | 20159 |
| 1 | 1 | 3 | Detection control | 62895 | 21744 | 61580 | 20100 |
| 1 | 1 | 4 | Detection control | 63392 | 20661 | 59309 | 20049 |
| 1 | 1 | 5 | Detection control | 63280 | 19970 | 59427 | 19833 |
| 1 | 1 | 6 | Detection control | 61901 | 19542 | 61272 | 19556 |
| 1 | 2 | 1 | Negative ctl (Buffer) | 22904 | 22675 | 19896 | 20329 |
| 1 | 2 | 2 | Negative ctl (Buffer) | 24482 | 23298 | 20035 | 20101 |
| 1 | 2 | 3 | Macroglobulin | 40100 | 20668 | 32619 | 19983 |
| 1 | 2 | 4 | Macroglobulin | 38793 | 20338 | 33165 | 19709 |
| 1 | 2 | 5 | Albumin | 64244 | 19559 | 63921 | 19485 |
| 1 | 2 | 6 | Albumin | 64392 | 19409 | 63641 | 19468 |
| 1 | 3 | 1 | Bcl-2 | 25811 | 23857 | 21220 | 20569 |
| 1 | 3 | 2 | Bcl-2 | 22493 | 21377 | 21509 | 20802 |
| 1 | 3 | 3 | IS1 | 63230 | 19739 | 62921 | 20643 |
| 1 | 3 | 4 | IS1 | 62358 | 19695 | 62478 | 20280 |
| 1 | 3 | 5 | C-jun | 21117 | 19849 | 22382 | 19895 |
| 1 | 3 | 6 | C-jun | 21818 | 20188 | 23523 | 20067 |
| 1 | 4 | 1 | C/EBP | 42424 | 23311 | 31394 | 20359 |
| 1 | 4 | 2 | C/EBP | 42833 | 20870 | 31754 | 20203 |
| 1 | 4 | 3 | Cox-2 | 20446 | 20125 | 20187 | 20156 |
| 1 | 4 | 4 | Cox-2 | 20429 | 20077 | 20484 | 20290 |
| 1 | 4 | 5 | Cyclin D1 | 28064 | 19929 | 25036 | 20303 |
| 1 | 4 | 6 | Cyclin D1 | 29258 | 20600 | 25587 | 20470 |
| 1 | 5 | 1 | Cyp 3a | 46066 | 22357 | 63098 | 20180 |
| 1 | 5 | 2 | Cyp 3a | 43555 | 20284 | 63114 | 19763 |
| 1 | 5 | 3 | Cyp 4a1 | 46866 | 19397 | 35089 | 19758 |
| 1 | 5 | 4 | Cyp 4a1 | 46995 | 19356 | 35673 | 20010 |
| 1 | 5 | 5 | HGPT | 33294 | 19275 | 26564 | 20006 |
| 1 | 5 | 6 | HGPT | 35003 | 20321 | 27506 | 20275 |
| 1 | 6 | 1 | Pos. Hyb. ctl. | 61592 | 22610 | 61310 | 20552 |
| 1 | 6 | 2 | Pos. Hyb. ctl. | 59717 | 19848 | 61215 | 20355 |
| 1 | 6 | 3 | Cyt oxidase 1 | 64045 | 18920 | 63947 | 20215 |
| 1 | 6 | 4 | Cyt oxidase 1 | 63527 | 19051 | 62432 | 20194 |
| 1 | 6 | 5 | Erk-1 | 23696 | 19093 | 22998 | 20448 |
| 1 | 6 | 6 | Erk-1 | 24348 | 19337 | 23360 | 20620 |
| 1 | 7 | 1 | ACO | 59718 | 20174 | 51686 | 21037 |
| 1 | 7 | 2 | ACO | 58672 | 19356 | 51619 | 20658 |
| 1 | 7 | 3 | GADD153 | 23974 | 19442 | 23334 | 20677 |
| 1 | 7 | 4 | GADD153 | 23662 | 19694 | 23216 | 20794 |
| 1 | 7 | 5 | IS2 | 61664 | 19389 | 61779 | 20644 |
| 1 | 7 | 6 | IS2 | 63181 | 19197 | 60083 | 20452 |
| 1 | 8 | 1 | GADD45 | 24674 | 21380 | 22359 | 21598 |
| 1 | 8 | 2 | GADD45 | 22313 | 20443 | 22287 | 21162 |
| 1 | 8 | 3 | Myr | 28927 | 20033 | 22905 | 21051 |
| 1 | 8 | 4 | Myr | 28139 | 20042 | 22579 | 21003 |
| 1 | 8 | 5 | GSH reductase | 25670 | 20065 | 24281 | 21085 |
| 1 | 8 | 6 | GSH reductase | 24960 | 20189 | 23937 | 21280 |
| 1 | 9 | 1 | Hox2 | 36516 | 20724 | 30000 | 21895 |
| 1 | 9 | 2 | Hox2 | 35432 | 20223 | 29975 | 21648 |
| 1 | 9 | 3 | HGF | 21114 | 20145 | 21195 | 21422 |
| 1 | 9 | 4 | HGF | 20926 | 20072 | 20996 | 21458 |
| 1 | 9 | 5 | Negative Hyb Ctl | 20401 | 20206 | 21211 | 21450 |
| 1 | 9 | 6 | Negative Hyb Ctl | 20728 | 20396 | 21255 | 21327 |
| 1 | 10 | 1 | IKB | 21600 | 21045 | 21781 | 22023 |
| 1 | 10 | 2 | IKB | 21345 | 20837 | 21227 | 21636 |
| 1 | 10 | 3 | MnSOD | 33717 | 20333 | 26695 | 21421 |
| 1 | 10 | 4 | MnSOD | 32706 | 20189 | 26251 | 21400 |
| 1 | 10 | 5 | NFKB | 21492 | 20191 | 21439 | 21447 |
| 1 | 10 | 6 | NFKB | 21681 | 20219 | 21343 | 21092 |
| 1 | 11 | 1 | P53 | 33024 | 21529 | 25848 | 23030 |
| 1 | 11 | 2 | P53 | 32417 | 21509 | 25221 | 22459 |
| 1 | 11 | 3 | PCNA | 23663 | 20379 | 23374 | 22205 |
| 1 | 11 | 4 | PCNA | 22944 | 19883 | 23609 | 22420 |
| 1 | 11 | 5 | Phospho A2 | 20496 | 19916 | 21966 | 22143 |
| 1 | 11 | 6 | Phospho A2 | 20509 | 19969 | 21421 | 21581 |
| 1 | 12 | 1 | MDR 1 | 31711 | 20633 | 27172 | 23658 |
| 1 | 12 | 2 | MDR 1 | 30443 | 20587 | 26841 | 22635 |
| 1 | 12 | 3 | Smp30 | 56588 | 19931 | 32696 | 23203 |
| 1 | 12 | 4 | Smp30 | 54764 | 19571 | 31894 | 23036 |
| 1 | 12 | 5 | Telomerase | 22317 | 19588 | 23256 | 22407 |
| 1 | 12 | 6 | Telomerase | 22564 | 19840 | 22745 | 21981 |
| 1 | 13 | 1 | IS3 | 59017 | 20875 | 49148 | 23658 |
| 1 | 13 | 2 | IS3 | 58812 | 20459 | 49118 | 23594 |
| 1 | 13 | 3 | Tubulin | 43799 | 20243 | 36262 | 24555 |
| 1 | 13 | 4 | Tubulin | 44979 | 20046 | 36054 | 24029 |
| 1 | 13 | 5 | UDPGT1a | 52864 | 19525 | 52205 | 22835 |
| 1 | 13 | 6 | UDPGT1a | 56082 | 19571 | 50981 | 21994 |
| 1 | 14 | 1 | Neg. Hyb. ctl. | 21506 | 20913 | 24703 | 24456 |
| 1 | 14 | 2 | Neg. Hyb. ctl. | 21836 | 20855 | 24372 | 25955 |
| 1 | 14 | 3 | Detection ctl. (conc. Curve) | 55228 | 20263 | 51864 | 25735 |
| 1 | 14 | 4 | Detection ctl. (conc. Curve) | 59905 | 20231 | 56090 | 24149 |
| 1 | 14 | 5 | Detection ctl. (conc. Curve) | 61037 | 20128 | 59154 | 23384 |
| 1 | 14 | 6 | Detection ctl. (conc. Curve) | 62439 | 19790 | 61112 | 23043 |
| 2 | 1 | 1 | Detection control | 63768 | 19662 | 60692 | 19572 |
| 2 | 1 | 2 | Detection control | 64099 | 20178 | 61330 | 19596 |
| 2 | 1 | 3 | Detection control | 63958 | 19972 | 60966 | 19779 |
| 2 | 1 | 4 | Detection control | 64057 | 20120 | 60157 | 19950 |
| 2 | 1 | 5 | Detection control | 63482 | 20075 | 61538 | 20150 |
| 2 | 1 | 6 | Detection control | 63413 | 20368 | 61663 | 20521 |
| 2 | 2 | 1 | ApoJ | 57551 | 19492 | 52452 | 19701 |
| 2 | 2 | 2 | ApoJ | 58419 | 19882 | 55866 | 20171 |
| 2 | 2 | 3 | B-actin | 58360 | 19895 | 52992 | 20570 |
| 2 | 2 | 4 | B-actin | 60490 | 19792 | 55039 | 21534 |
| 2 | 2 | 5 | Bax | 25797 | 20149 | 24767 | 22102 |
| 2 | 2 | 6 | Bax | 28007 | 21070 | 27814 | 23975 |
| 2 | 3 | 1 | Neg. Hyb. ctl. | 20781 | 20393 | 21630 | 19985 |
| 2 | 3 | 2 | Neg. Hyb. ctl. | 21061 | 20348 | 22233 | 21056 |
| 2 | 3 | 3 | IS1 | 62029 | 19919 | 58872 | 22005 |
| 2 | 3 | 4 | IS1 | 62862 | 19804 | 59444 | 23271 |
| 2 | 3 | 5 | C-myc | 21412 | 20401 | 27229 | 25377 |
| 2 | 3 | 6 | C-myc | 22247 | 21392 | 28315 | 23722 |
| 2 | 4 | 1 | Cyp 1a1 | 21570 | 20937 | 20728 | 20377 |
| 2 | 4 | 2 | Cyp 1a1 | 21579 | 20770 | 20972 | 20731 |
| 2 | 4 | 3 | Gyp 1b1 | 20928 | 20329 | 22231 | 20962 |
| 2 | 4 | 4 | Cyp 1b1 | 20635 | 20184 | 21071 | 20894 |
| 2 | 4 | 5 | Cyp 2b | 31072 | 20423 | 62971 | 20928 |
| 2 | 4 | 6 | Cyp 2b | 31910 | 20859 | 62867 | 20497 |
| 2 | 5 | 1 | Elk | 20303 | 19936 | 20632 | 20274 |
| 2 | 5 | 2 | Elk | 20920 | 19871 | 20630 | 20397 |
| 2 | 5 | 3 | Enoyl CoA | 61062 | 19769 | 60758 | 20263 |
| 2 | 5 | 4 | Enoyl CoA | 58376 | 19657 | 60738 | 20101 |
| 2 | 5 | 5 | Neg. Hyb. ctl. | 20318 | 19951 | 20235 | 20093 |
| 2 | 5 | 6 | Neg. Hyb. ctl. | 20734 | 20414 | 20288 | 20116 |
| 2 | 6 | 1 | Ferritin | 60679 | 18959 | 60237 | 20182 |
| 2 | 6 | 2 | Ferritin | 61070 | 18971 | 59913 | 20019 |
| 2 | 6 | 3 | Fibronectin | 53853 | 19698 | 47542 | 20010 |
| 2 | 6 | 4 | Fibronectin | 50994 | 19887 | 50240 | 20182 |
| 2 | 6 | 5 | GAPDH | 48633 | 20365 | 58466 | 20401 |
| 2 | 6 | 6 | GAPDH | 53398 | 20007 | 60437 | 20126 |
| 2 | 7 | 1 | Glutatione Ya | 63382 | 19272 | 63398 | 19910 |
| 2 | 7 | 2 | Glutatione Ya | 64142 | 19152 | 63872 | 19871 |
| 2 | 7 | 3 | Glutathione Theta 5 | 34346 | 19683 | 30062 | 20209 |
| 2 | 7 | 4 | Glutathione Theta 5 | 35564 | 20312 | 32113 | 20815 |
| 2 | 7 | 5 | IS2 | 57626 | 20108 | 58852 | 20935 |
| 2 | 7 | 6 | IS2 | 51367 | 20073 | 60594 | 20775 |
| 2 | 8 | 1 | Histone Dacetyl | 22688 | 20410 | 21846 | 20810 |
| 2 | 8 | 2 | Histone Dacetyl | 22719 | 20359 | 21766 | 20760 |
| 2 | 8 | 3 | HMG | 50616 | 20223 | 34978 | 21077 |
| 2 | 8 | 4 | HMG | 50319 | 20482 | 36070 | 21339 |
| 2 | 8 | 5 | Hsp 70 | 21733 | 20676 | 23738 | 21494 |
| 2 | 8 | 6 | Hsp 70 | 22156 | 20672 | 22628 | 21625 |
| 2 | 9 | 1 | II6 | 20620 | 20446 | 20776 | 20956 |
| 2 | 9 | 2 | II6 | 24332 | 20445 | 20646 | 20775 |
| 2 | 9 | 3 | JNK | 20898 | 20059 | 20743 | 20798 |
| 2 | 9 | 4 | JNK | 20902 | 20242 | 20935 | 20767 |
| 2 | 9 | 5 | Mgmt | 27289 | 20100 | 23373 | 20859 |
| 2 | 9 | 6 | Mgmt | 27256 | 20182 | 23981 | 21023 |
| 2 | 10 | 1 | ODC | 25227 | 19644 | 23381 | 20590 |
| 2 | 10 | 2 | ODC | 24891 | 19811 | 23303 | 20605 |
| 2 | 10 | 3 | Pos Hyb ctl | 63131 | 19540 | 60888 | 20536 |
| 2 | 10 | 4 | Pos Hyb ctl | 62019 | 19531 | 60017 | 20394 |
| 2 | 10 | 5 | P38 | 37070 | 19729 | 26227 | 20684 |
| 2 | 10 | 6 | P38 | 35832 | 20047 | 28841 | 21213 |
| 2 | 11 | 1 | Ubiquitin | 61744 | 19935 | 59171 | 20578 |
| 2 | 11 | 2 | Ubiquitin | 62298 | 19754 | 59613 | 20252 |
| 2 | 11 | 3 | PPAR | 21687 | 19506 | 20767 | 20480 |
| 2 | 11 | 4 | PPAR | 21830 | 19780 | 20704 | 20377 |
| 2 | 11 | 5 | S29 | 61135 | 19888 | 50843 | 20264 |
| 2 | 11 | 6 | S29 | 60757 | 20004 | 53202 | 20456 |
| 2 | 12 | 1 | TNF | 20553 | 19940 | 21082 | 21210 |
| 2 | 12 | 2 | TNF | 20370 | 19928 | 20767 | 20960 |
| 2 | 12 | 3 | Transferrin | 63801 | 19982 | 61525 | 21193 |
| 2 | 12 | 4 | Transferrin | 63226 | 20131 | 61527 | 21002 |
| 2 | 12 | 5 | TGFbRII | 24493 | 20012 | 22149 | 21099 |
| 2 | 12 | 6 | TGFbRII | 20328 | 19970 | 23021 | 22027 |
| 2 | 13 | 1 | IS3 | 55666 | 19977 | 45025 | 21756 |
| 2 | 13 | 2 | IS3 | 55285 | 20352 | 43614 | 21829 |
| 2 | 13 | 3 | UDPGT1a6 | 22207 | 20978 | 22769 | 22436 |
| 2 | 13 | 4 | UDPGT1a6 | 22324 | 20919 | 22766 | 22354 |
| 2 | 13 | 5 | Neg. Hyb. ctl. | 20831 | 20420 | 21348 | 22027 |
| 2 | 13 | 6 | Neg. Hyb. ctl. | 21178 | 20992 | 21647 | 21893 |
| 2 | 14 | 1 | Negative ctl (Buffer) | 22367 | 21281 | 23233 | 23184 |
| 2 | 14 | 2 | Negative ctl (Buffer) | 22429 | 22401 | 23115 | 23166 |
| 2 | 14 | 3 | Detection ctl. (conc. Curve) | 55098 | 22598 | 48150 | 23322 |
| 2 | 14 | 4 | Detection ctl. (conc. Curve) | 59678 | 21765 | 54485 | 22852 |
| 2 | 14 | 5 | Detection ctl. (conc. Curve) | 60802 | 21263 | 58019 | 21450 |
| 2 | 14 | 6 | Detection ctl. (conc. Curve) | 58129 | 21843 | 61419 | 21231 |

The Rat HepatoChips is composed of single strand DNA probes attached to the glass by a covalent link. The length of the DNA nucleotide sequences has been optimized. They are the same for all genes and are located near the 3′ end of the transcript. All probes have been designed to be gene specific and have been prepared using rat cDNAs Two spots per gene have been spotted onto the array, except for some of the control probes.

Synthesis of Labeled cDNA

Labeled CDNA was prepared using 2 μg mRNA isolated using the FastTrack 2.0 mRNA isolation Kit (Invitrogen). A synthetic poly (A)+tailed mRNA was spiked to the purified mRNA as internal standard to assist in quantification and estimation of experimental variation introduced during labeling and reading. mRNA was added to 2 μl of oligo dT(12-18) primer (0.5 μg/ul) (Gibco BRL), RNase free water was used to bring the volume to 9 μl, and the mixture was denatured at 70° C. for 10 min and then chilled on ice for 5 min. The reverse transcription was performed by adding the following components to the annealed probe/template on ice: 4 μl of First Strand Buffer (250 mM Tris-HCl pH 8.3, 375 mM KCl, 15 mM MgCl2) (Gibco BRL), 2 ul of DTT 0.1M (Gibco BRL), 40 units of RNasin ribonuclease inhibitor (Promega), 500μM dATP (Roche), 500 μM dTTP (Roche), 500 μM dGTP (Roche), 80 μM dCTP (Roche), 80 μM biotin-11-dCTP (NEN). The reaction mixture was mixed gently by flicking the tube and incubated for 5 min at room temperature. 300 units of SuperScript II RT (RNase H-) (Gibco BRL) was added to the reaction mixture and the reverse transcription was allowed to proceed for 90 min at 42° C. Then an additional 300 units of SuperScript II RT was added and incubation was continued at 42° C. for another 90 min. The reaction was ended by heat inactivation at 70° C. for 15 min. To remove RNA complementary to the cDNA, a treatment with RNase H was performed at 37° C. for 20 min following by a heat denaturation at 95° C. for 3 minutes and cooled on ice before use. No further RT product purification was necessary.

Hybridization Using Biotinylated cDNA

The hybridization was performed in a hybridization chamber (Biozym, Landgraaf, The Netherlands) containing the hybridization buffer ‘Hepatobuffer’ and a positive hybridization control (a biotinylated amplicons, at a concentration of 25 nM). Hybridization was carried out overnight at 60° C. The arrays were then washed four times for 2 min with washing buffer at room temperature.

Colorimetric Silver Detection

The presence of biotinylated hybrids on the microarray was detected using a antibody anti-biotin conjugate coupled colloidal gold. The arrays were then incubated with a 1:100 dilution of conjugate solution in a blocking buffer (100 mM maleic buffer pH 7.5, 150 mM NaCl and 0.1% milk powder) for 45 min at room temperature. The array were then washed five times for 2 min at room temperature, rinsed briefly with deionized water then dried. Then array is incubated at room temperature for 5 min in the Silver Blue Solution (AAT, Namur, Belgium), rinsed in water, dried 5′ at 37° C. and read with the scanner described herein.

Scanning Device

The scanner used had the following scanning parameters:

Bit depth: 16 bits grayscale (65536 grey levels)

no additional correction of the image (i.e. standard values for contrast, brightness, . . . )

Scanning software: Silverfast from LaserSoft

Quantification Software: Imagene 4.2 from Biodiscovery

Results

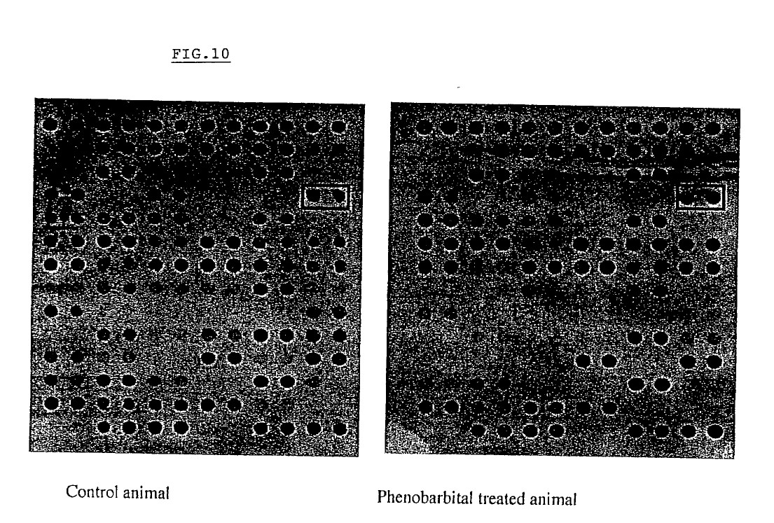

The scanning images clearly already show some differentially expressed genes. The raw quantitative scanned data are given in the Table 1. For each spot of each array, the quantification software gives the values of the spot intensity mean and the local background, in 16-bits grayscale (from 1 to 65536). A digitalized picture is presented in FIG. 10 for illustration.

### EXAMPLE 3

### Detection of Proteins on Biochips

Fixation of Antibodies on the Array

The glass of the array was activated as described here above in order to obtain aldehyde groups on the surface. The antibodies used in this experiment were raised against bovine serum albumin for positive control and non specific IgG for negative control. The antibodies at 10 μg/ml in PBS solution were spotted using the 250 μm diameter pins directly on the glass. The amino groups of the antibodies could react with the aldehyde present on the glass. The reaction was performed for 1 h at room temperature. The gasses were washed with a PBS buffer.

Detection of Bovine Serum Albumin by ELISA on the Array

A solution of bovine serum albumin (BSA) at 10 μg/ml in PBS containing 0.1% casein was added on the array and incubated for 30 min. The array was then washed 3 times with PBS containing 0.1% Tween 20 and then incubated with a solution of biotinylated anti-BSA at 20 μg/ml in PBS containing 0.1% casein. The incubation was performed for 30 min. A streptavidin-Gold complex at 1 μg/ml was then incubated for 30 min in a PBS solution containing 0.1% casein. The presence of gold served as a center for silver reduction. The silver precipitation was performed with a “silver enhancement reagent” from Sigma with a change of the solution after 10 min and then again after 5 min. The glasses were then scanned and the data analyzed as presented in the example here above.

### EXAMPLE 4

### Method for Detection of IgE by ELISA on Microarrays and Calorimetric Detection

The sandwich detection was performed as follows: RAT IgE antibodies (from mouse) were spotted on aldehyde slides (Diaglass, AAT, Namur, Belgium) in a spotting buffer (AAT, Namur, Belgium). The spotting was obtained with solid pins of 0.250 mm diameter and the spots were around 0.35 mm diameter final After 4 washes of 2 minutes with phosphate pH 7.4 0.01M+0.1% Tween 20, non-specific binding sites were blocked with maleate buffer 100 mM pH 7.5 containing 150 mM NaCl milk powder at 0.1% (blocking buffer) for 1 h at 20° c. The slides chambers were incubated for 1 h at 20° c. with RAT IgE (diluted 10 000 times in blocking buffer). After 4 washes of one minute with a 10 mM maleate buffer containing 15 mM NaCl and 0.1% Tween pH 7.5 (washing buffer) the slides were incubated for 1 h with RAT IgE antibodies (from GOAT) (diluted 1000 times). After 4 washes of one minute with a 10 mM maleate buffer containing 15 mM NaCl and 0.1% Tween pH 7.5 (washing buffer) slides were incubated for 45 min at 20° c. with a anti-GOAT-IgG conjugate to gold nanoparticules of 20 nm diameter (diluted 100 times) in blocking buffer.

Slides were washed 4 times (for 2 minutes) in the same washing buffer as before and then incubated for 10 min in the Silver Blue detection solution (AAT, Namur) for obtaining the silver crystal precipitation.

### EXAMPLE 5

### Detection of Auto-Immune Antibodies

Applications on the detection of autoimmune disease by the identification of the antibodies is very well adapted to the protein chips on glass slides since a large number of possible antibodies can be screened simultaneously for their possible presence in the patients fluids. These included the detection of the anti-neutrophil-cytoplasmic antibodies (ANCA) such as the Proteinase 3(PR3) for the diagnostic of the Wegener's granulomatosis, the Myeloproxidase (MPO)for the diagnostic of the Churg-Strauss syndrome, polyarteritis nodosa, microscopic polyangiitis and Rapid Progressive Glomerulonephritis. Other autoantibodies useful to detect are the anti-cell nuclei (ANA) (mRNP/Sm, SM,SS-A,SS-B,Scl-70), the anti-mitochondria (AMA), the anti-liver antigens, the anti-Parietal Cells (PCA), the anti-Neuronal Antigens (Hu,Yo,Ri), the anti-endomysium.

Other applications are the detection of different antibodies as anti-thyroglobulines, anti-thyroperoxidases, the anti-insulin, anti-erythrocytes, anti-gliadine, anti-HLA A,B,C and DR, anti-thrombocytairs, anti-tissue, anti-spermatozoids, anti-nuclear, anti-cytoplasmic antibodies. In diabetes, useful assays are the detection autoantibodies such as IA-2 autoantibodies, the anti-Islet Cell antibodies (ICA), the anti-insulin antibodies (IAA)and the anti-GAD antibodies.

The experiment was performed as described in example 3. The exact procedure was as followed:

Antigens were spotted on the aldehyde activated glass slide (DIAGLASS slides, AAT, Namur, Belgium) The antigens spotted on the slide were: La(SSN) Ag, JO-1 Ag, Scl-70 Ag, RNP/Sm Ag, Ro(SSA) Ag. Protein A gold was used as a positive control for detection, mouse antibody and streptavidin used as negative controls The antigens were diluted to a final concentration of 100 μg/ml in a spotting buffer (AAT, Namur, Belgium) and spotted as an antigen at the surface of an aldehyde based polymer coated glass slide as explained in example 3. For detection of antibodies, the slides were incubated for 1 h at 20° c. with different human sera diluted to 1/100 in the blocking buffer. After 4 washes of one minute with a 10 mM maleate buffer containing 15 mM NaCl and 0.1% Tween pH 7.5 (washing buffer) slides were incubated for 45 min at 20° c. with a conjugate of anti-human IgG(H+L)/gold particles of 10 nm diameter (diluted 100 times) in 100 mM blocking buffer.

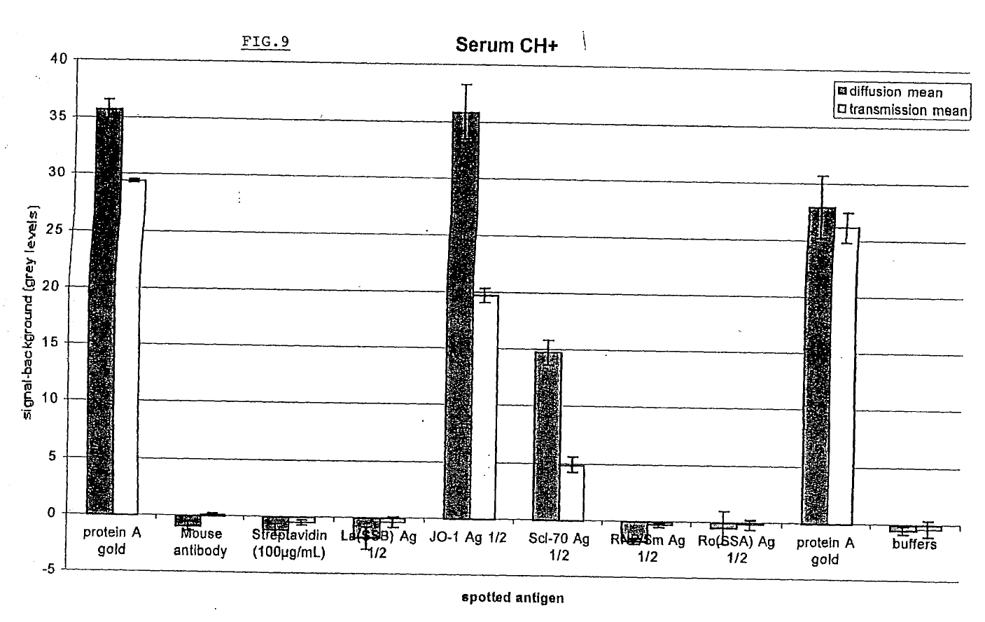

Slides were washed 4 times (for 2 minutes) in the same washing buffer as before and then incubated for 10 min in the Silver Blue detection solution (AAT Namur) for obtaining the silver crystal precipitation. The slides were finally washed in distilled water before being read in the scanner and quantified using Imachips software (WOW Company). One result for Serum CH+ is showed in FIG. 9. We can observe a positive fixation of the JO-1 and Scl-70 Ag antigens. This was confirmed by the ELISA assays. However, the reaction on the Scl-70 Ag is weak and can be easily obtained with the diffusion method while it is not significantly detected with the transmission detection.

### EXAMPLE 6

### Detection of Multiple Microarrays Handling in an Automate

Microarrays were constructed on a surface of polypropylene coated with a methylacrylate and polymerized by irradiation under UV light. The capture molecules (nucleotide probes) were spotted as explained in the example 4. The surface of the polycarbonate was 12.8×8.5 cm. 4×6 arrays were spotted on the surface in a rectangular pattern with a distance of 18 mm between the center of each array. The arrays were surrounded by hybridization chambers cut according to the arrays pattern in a double coated polymer covering the overall surface of the support. The arrays locations were excentric compared to the pipettes in order to pipet solutions on the side of the chambers. The support was inserted into a laboratory automation workstation Biomek© 2000 (Beckman Coulter). The automate was used in conjunction with several interchangeable tools for adjusting the liquid delivered (between 0.05 and 0.2 ml). The automate was controlled by a IBM Pentium-based computer with seven communication ports using the software controller, BioWorks 3.0 from Beckman Coulter. The robot possesses robotic arms with a 8 pipettes support. The position of the arm above the plate has a precision of around 0.01 mm.

After incubation, the washing solutions and the reagents for conjugate, silver labeling were delivered and removed from the hybridization chambers by the automate. A digitalized picture of each of the arrays were taken by a CCD camera and processed for analysis.

### EXAMPLE 7

### Retro-Diffusion Device (Scanning Means Combining Transmission and Diffusion Mode Light for Increasing Scanner Dynamic Range

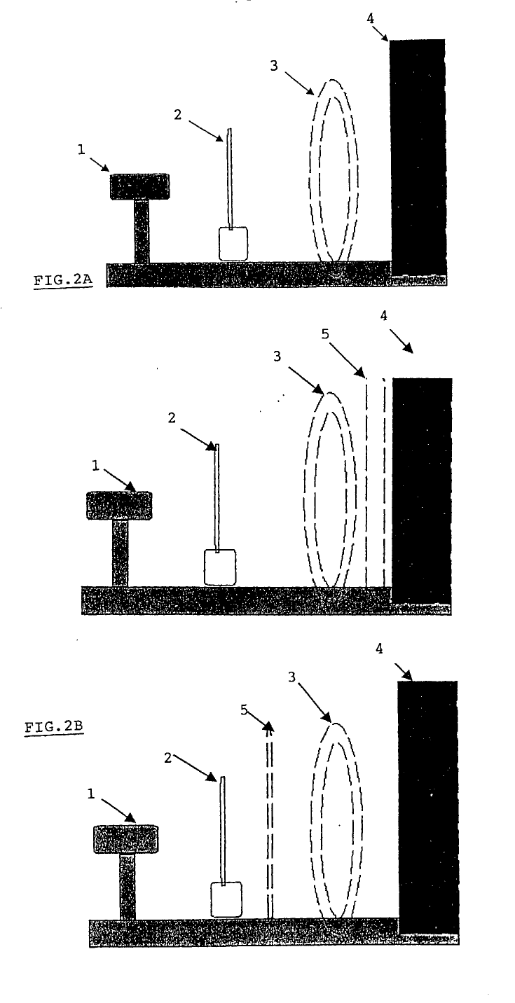

Hardware: cf. FIG. 2

Optical bench comprising a circular neon light tube (3) (Hg, 100 kHz, controlled and stabilized).

A detector (1) CCD camera (from Creative, 8 bits), a black support surface (4) for dark background and white support surface (5) for white background.

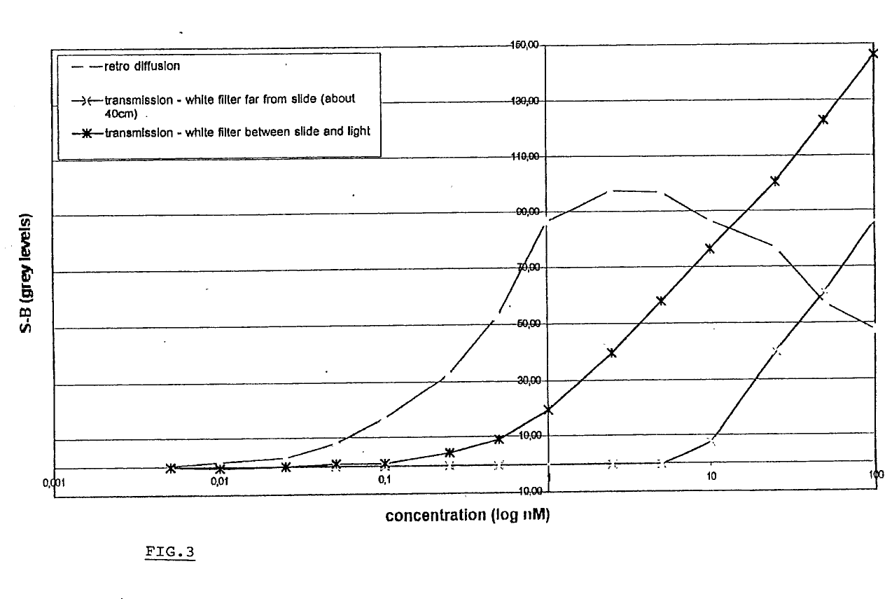

Silver Blue TM revealed biochips (2) (obtained from AAT Belgium) with biotinylated CMV DNA concentration curves. Type of array used: 9×6 concentration curve (see FIG. 3). Software:

Imachips 1.08 obtained from WOW Company in Belgium for image quantification.

The same slides were scanned several times using different configurations on the optical bench: 101491 retro-diffusion mode (FIG. 2a): CCD camera (1), slide sample (2), circular neon tube (3) and black background (4) placed after the neon tube

transmission mode (FIG. 2b): CCD camera (1), slide sample (2), circular neon tube (3), black background (4), and/or white support surface (5) placed either between light and slide or between light and black background.

The output image was quantified by Imachips software.

TABLE 2
1. Values obtained by transmission or retro-diffusion from the
measurements of the same spotted DNA on microarrays

| | | Transmission | |
|---|---|---|---|
| | | white support | Transmission |
| Concentration | | surface between | white filter |
| (nM) of spotted | Retro diffusion | slide and black | between slide |
| solution | (Black background) | background | and light |
| 0.005 | 0.17 | 0.00 | −0.33 |
| 0.01 | 1.70 | 0.00 | −0.77 |
| 0.025 | 3.07 | 0.00 | −0.33 |
| 0.05 | 8.40 | 0.00 | 0.87 |
| 0.1 | 17.30 | 0.00 | 0.63 |
| 0.25 | 32.60 | 0.00 | 4.47 |
| 0.5 | 53.07 | 0.00 | 9.03 |
| 1 | 86.83 | 0.00 | 19.37 |
| 2.5 | 97.43 | 0.00 | 39.67 |
| 5 | 97.23 | 0.00 | 57.87 |
| 10 | 86.67 | 7.63 | 76.53 |
| 25 | 77.20 | 39.97 | 100.63 |
| 50 | 56.90 | 61.27 | 122.70 |
| 100 | 47.60 | 86.47 | 146.17 |

The intensity values of table 2 are means of the triplicates measurements for each concentration. The values are given as Intensity=Signal−Local Background

The present invention is based upon a new concept of detection in addition to reflection/diffusion and transmission named hereafter “retro-diffusion” when the glass slide (2) is between the camera (1) and the neon light (3).

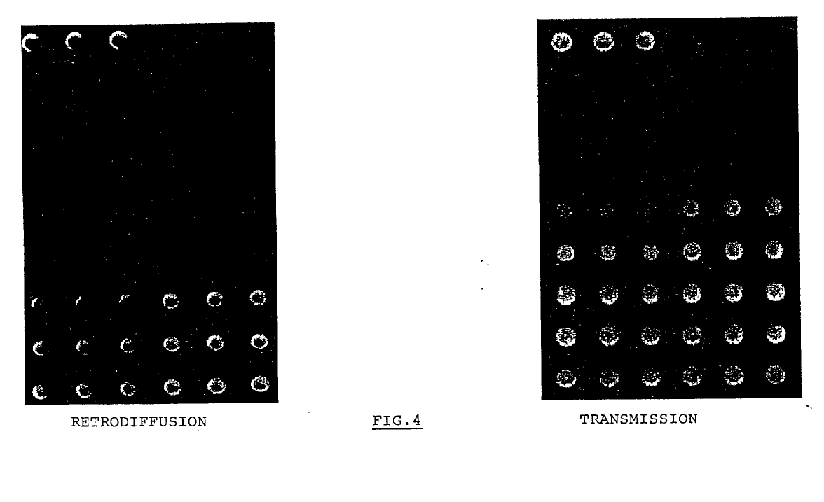

The left picture of FIG. 4 shows that the sensitivity, considered as the lowest concentrations detected, is higher than on the right picture of FIG. 4 and is able to detect a discrimination between low concentration spots.

The saturation in the high concentrations of the diffusion can be compensated by taking a picture using a white background (transmission measurement).

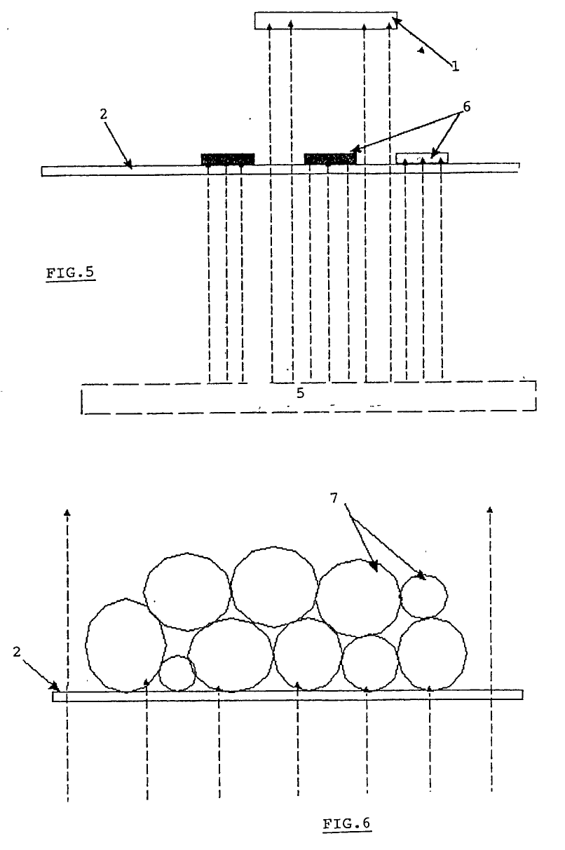

The difference between the two phenomena is explained in reference to the FIGS. 5 to 8.

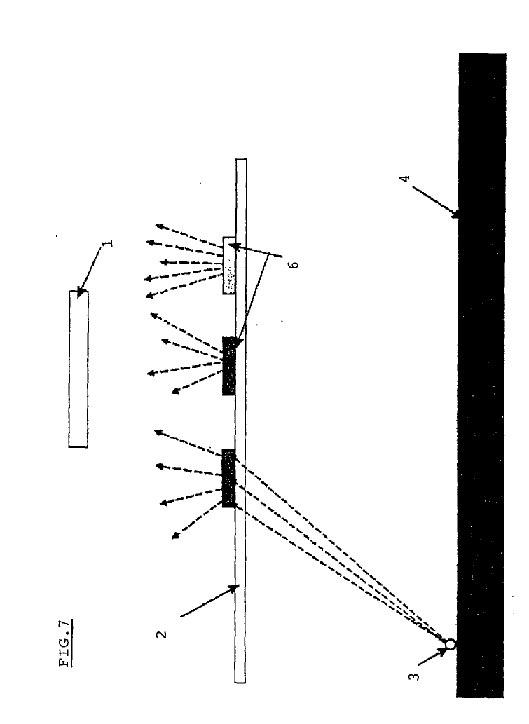

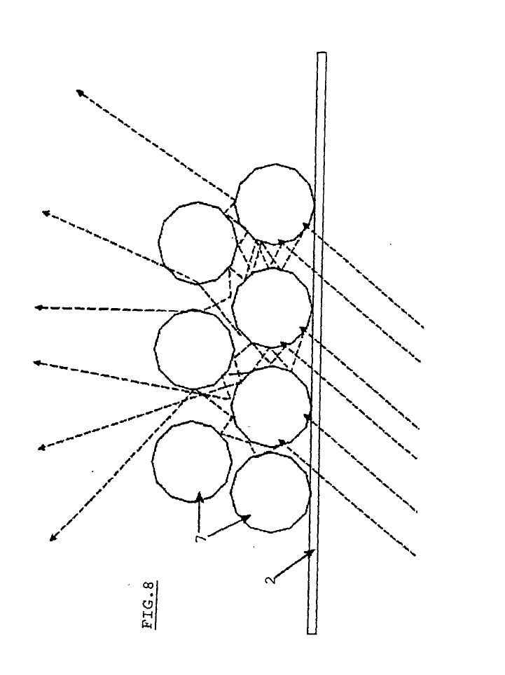

In retro-diffusion, the light goes through the silver crystals spots (5) that diffuse the light. The spots appear white on the black background (4) (FIG. 7). In retro-diffusion, at low concentration, there is spaces between the silver crystal, allowing multiple reflection and diffusion of the light (FIG. 8). The method is well adapted for measurement of the low concentrations while at high concentrations, diffusion of the light beams is inhibited and signal intensity decreased.

In transmission, the light is absorbed and blocked by the metal particles or crystal silver crystals spot (FIG. 5). The absorption of light waves at very low concentrations is low compared to the light beam intensity and the measurement is not sensitive. At high concentrations however, the absorption allows good quantification of the signal (FIG. 6).

Combining the two methods allows to compensate the non-efficiency of the diffusion signal at high concentrations and the non accurate sensitivity of the transmission method at low concentrations. In this way a very large dynamic range using two pictures of the same slide with the same detector (camera) can be obtained. The concentration range of the detection goes from 0.01 nM to 100 nM (log 100/0.01)=4 logs.

Using a matrix CCD sensor gives images that can be perfectly and rapidly superimposed as only the white surface is moved between the two pictures acquisition.

In order to cover this whole dynamic range, the software used in conjunction with the scanner was developed in order to reconstruct one single curve from the two pictures.

The Examples described above are set forth solely to assist in the understanding of the invention. One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the following claims.

What is claimed is:

1. A method for an identification and/or the quantification of a target compound obtained from a sample, preferably a biological sample, comprising the steps of:
(a) putting into contact the target compound with a capture molecule in order to allow a specific binding between said target compound with a capture molecule, said capture molecule being fixed upon a surface of a solid support according to an array comprising a density of at least 20 discrete regions per cm², each of said discrete regions being fixed with one species of capture molecules;
(b) performing a reaction leading to a metallic precipitate formed at the location of said binding;
(c) determining the possible presence of precipitate(s) in discrete region(s); and
(d) correlating the presence and/or the formation of the precipitate(s) at the discrete region(s) with the identification and/or a quantification of said target compound.

2. The method of claim 1, wherein said metallic compound is a magnetic metallic compound.

3. The method of claim 1, wherein the reaction leading to the formation of the metallic precipitate is an enzymatic reaction.

4. The method of claim 1, wherein the reaction leading to the formation of the precipitate is a chemical reduction of silver in the presence of colloidal gold particles coupled to the bounded target compound.

5. The method of claim 1, wherein the specific binding between the target compound and its corresponding capture molecule is a hybridization between two nucleotide sequences.

6. The method according claim 1, wherein the capture molecule is a protein.

7. The method according claim 1, wherein the binding between the target compound and its corresponding capture molecule is a binding between an antigenic structure and its corresponding antibody or a hypervariable portion thereof.

8. The method of claim 1, wherein the binding between the target compound and its corresponding capture molecule is a binding between a receptor and its corresponding ligand.

9. The method of claim 5, wherein the capture molecule is a nucleotide sequence spotted on the surface of the solid support in the presence of a polyol.

10. The method of claim 6, wherein the target compound is identified and/or quantified according to a signal characteristic of a cell activation.

11. The method of claim 6, wherein the target compound is identified and/or quantified by a detection of a presence or a formation of a phosphate, an acetyl or a methyl group upon the target compound.

12. The method of claim 6, wherein the target compound is detected through a direct or indirect interaction with one or more molecules bound to the solid support.

13. The method of claim 1, wherein the possible presence of a precipitate is obtained by reflection, absorption or diffusion of a light beam upon said precipitate.

14. The method of claim 13, wherein the light beam is a laser beam.

15. The method of claim 1, wherein the presence of a precipitate in a discrete region is obtained by variation of an electromagnetic field or the conductance of an electric current.

16. The method of claim 1, wherein the step for determining a possible presence of a precipitate in a discrete region of the solid support surface is obtained by projecting a transmission light from a first light source onto said surface during a transmission mode time period, on a detector from said first light source which has been transmitted through said solid support and projecting diffuse scattering light from the same light source or a second light source onto said solid support during similar or other than said reflection mode time period and detecting reemitted light on said detector from said solid support surface.

17. The method of claim 1, which is a high-throughput screening identification and/or quantification method of target compound(s) upon corresponding capture molecules, wherein the solid support comprises between 4-well and 1536-well arrays disposed according to a multiple well microtitre plate format.

18. The method of claim 17, wherein said multiple wells microtitre plate is selected from the group consisting of 24-well, 96-well, 384-well and 1536-well microtitre plate formats.

19. The method of claim 1, wherein the support bearing capture molecules is inserted or is part of a pipette.

20. The method of claim 1, wherein the array present on the solid support surface is washed by a series of pipette(s) being disposed on an arm and moving according to X/Y axes of said solid support surface.

21. The method of claim 1, wherein the amount of precipitates corresponding to a detected and/or quantified target compound is compared to at least two different arrays.

22. The method of claim 21, wherein a target compound amount detected in a tested sample which statistically differ from a reference sample, represents a pattern of target compound typical of the tested sample.

23. A diagnostic and/or quantification apparatus of a target compound obtained from a sample comprising:
(a) a solid support with an array surface comprising at least 4 discrete regions per cm2/surface, each of said region being fixed with one species of capture molecule corresponding to a target compound;
(b) a detection and/or quantification device of a metallic precipitate upon the surface of said solid support and resulting in a binding of said target compound upon a corresponding capture molecule;
(c) possibly a reading device of information(s) recorded upon said solid support;
(d) a computer programmed to:
(1) possibly recognize the discrete regions bearing capture molecules,
(2) collect the results obtained from said detection and/or quantification device, possibly comparing with the information(s) obtained from said reading device; and
(3) carry out a detection and/or quantification of said target compound(s).

24. The apparatus of claim 23, wherein said solid support with an array surface comprises at least 10 discrete regions per cm²/surface.

25. The apparatus of claim 23, wherein said a solid support with an array surface comprises at least 20 discrete regions per cm²/surface.

26. The apparatus of claim 23, wherein the detection and/or the quantification device contains a detector with either a linear or a matricial sensor.

27. The apparatus of claim 26, wherein the detector of the transmitted or diffracted light is selected from the group consisting of one or more diode(s), a CCD camera, a CMOS camera and a fiber optic bundle.

28. Diagnostic and/or quantification apparatus of claim 23, wherein the detection and/or quantification device of precipitate comprises a first light source providing a transmission mode light to the solid support and a second light source or same light source providing diffuse scattering (diffraction) light to said solid support, a detector and a computer program to interact with said detector such that said detector detects light transmitted from said support in response to application of light from said first light source and said detector detected reemitted light in response to application of light from said second light source wherein said device is configured to cause the first light source to provide a transmission mode light to the solid support, preferably during a first time period, and to cause a second light source to provide diffuse scattering light to said sample preferably (during a time period other than said first time period).

29. The apparatus of claim 28, comprising a light source obtained from a circular neon tube, a black background (and possibly a white moveable translucent surface) disposed between the solid support and the source light or wherein the source light is disposed between the solid support and said white surface.

30. The apparatus of claim 23, which is a high-throughput screening apparatus, wherein the solid support surface comprises multiple arrays surfaces disposed according to a multiple well microtitre plate format.

31. The apparatus of claim 30, wherein the multiple wells microtitre plate format is selected from the group consisting of 36-well, 384-well and 1536-well microtitre plate formats.

32. The apparatus of claim 30, which further comprises an arm bearing the detection and/or quantification device which moves according to X/Y axes of the solid support surface, or multiple of it.

33. The apparatus of claim 30, wherein the detection is performed simultaneously or consecutively by a computer controlled moving device which analyses discrete regions bearing capture molecules present on the solid support surface and attribute the results obtained from the detection and/or quantification device to each sample initially introduced in the array surface.

34. The apparatus of claim 30, comprising an automatic liquid handling device for pipeting in the array surface.

35. The apparatus of claim 32, wherein the device moves according to steps of 9 mm or multiple of it.

36. A computer program comprising program code means for performing the steps of determining the possible presence of a precipitate in discrete regions and correlating the presence of said precipitate at the discrete regions with the identification and/or the quantification of a target compound, according to the method of claim 1, when said program is run on a computer.

37. A computer program product comprising program code means stored on a computer readable medium for performing the steps of determining the possible presence of a precipitate in a discrete region and correlating the presence of the precipitate at the discrete region with the identification and/or the quantification of a target compound, according to the method of claim 1, when said program is run on a computer.
